(19) United States
(12) Patent Application Publication
(10) Pub. No.: US 2021/0077446 A1
(43) Pub. Date: Mar. 18, 2021

(54) PUFA SALT FORMULATIONS (II)

(71) Applicant: DSM IP ASSETS B.V., Heerlen (NL)

(72) Inventors: Elger FUNDA, Kaiseraugst (CH); Denis HUG, Kaiseraugst (CH); Odile KRAINZ, Kaiseraugst (CH); Sean MCDONNELL, Kaiseraugst (CH); Shakuntala RAMNARAIN, Kaiseraugst (CH); Casey SUMNER, Kaiseraugst (CH); Qiong TANG, Kaiseraugst (CH)

(21) Appl. No.: 16/468,423

(22) PCT Filed: Dec. 13, 2017
(86) PCT No.: PCT/EP2017/082552

§ 371 Date: Jun. 11, 2019

(30) Foreign Application Priority Data
Dec. 13, 2016 (EP) 16203600.8

(51) Int. Cl. A61K 31/202; A61K 9/16; A61K 47/34; A61K 47/26; A61K 47/22; A61K 47/06; A61K 47/10; A23L 33/12; A23L 33/18; A23L 33/125; A23L 3/46

(52) U.S. Cl. CPC A61K 31/202; A61K 9/1658; A61K 47/34; A61K 47/26; A61K 47/22; A23V 2002/00; A61K 47/10; A23L 33/12; A23L 33/18; A23L 33/125; A23L 3/46; A61K 47/06

(57) ABSTRACT

The present patent application relates to novel polyunsaturated fatty acid salt (PUFA salts) solid formulations.

The present patent application relates to novel polyunsaturated fatty acid salt (PUFA salts) solid formulations.

Polyunsaturated fatty acids (PUFAs) are very well-known compounds for a healthy diet (especially the omega-3 fatty acids). The PUFAs (especially the omega-3 fatty acids) have variety of health benefits against for example cardiovascular diseases (CVDs) including well-established hypotriglyceridemic and also against anti-inflammatory effects.

Therefore, PUFAs are important ingredients in a healthy diet (for animals and humans).

PUFAs can be found in a variety of plants and animal in various quantities and in various mixtures (of the different PUFA).

A very good source of omega-3 fatty acids are for example fish.

However, it is also possible to produce PUFAs synthetically.

Since many consumers do not like fish, especially the smell and taste of it, (or other PUFA containing sources), it is very common to add PUFAs to other dietary products (enrich these products with PUFA) to enable the desired healthy diet.

The problem of the PUFAs is, that they have strong tendency to oxidise. This results in a loss of the PUFAs in the product and secondly (even worse) in the development of a strong and very unpleasant smell.

With increasing number of double bonds, the PUFAs are subject to increasing oxidative degradation and development of undesirable “off-flavours”, mainly fishy and rancid smell and taste. Volatile degradation products cause off-flavour even at very low concentration. Sensory properties of a product may become unacceptable even before a loss of PUFAs can be detected analytically.

Another issue arises because PUFAs are oils compounds and therefore the incorporation of PUFA is not so easy and usually needs an emulsification step.

Now it was found that when PUFA salts (usually Na, K or Ca salts) are used instead of PUFA very stable and easy to handle solid formulations are obtained.

By the term “solid formulation”, it is meant that the formulation is in form. It is usually in the form of a powder, granule or beadlets. These formulations differ in the size of their particles.

Surprisingly it was found out that when at least one PUFA salt is embedded in a matrix comprising protein hydrolysate from a starch containing plant, a very stable solid formulation is obtained.

Therefore, the present invention relates to a solid formulation (SF) comprising
(i) at least one PUFA salt, and
(ii) at least one protein hydrolysate from a starch containing plant.

As stated above the solid formulation can have various particles sizes.

When the solid formulation is a spray dried powder form the preferred average particle size of the solid particle is 10-200 μm.

When the solid formulation is a beadlet, the preferred average particle size of the beadlet is 200-1000 μm.

When the solid formulation is a granule/pellet the preferred average particle size of the granule/pellet is preferably below 1000 μm.

The particle size is determined by using well-known methods, such as (scanning) electron microscopy. The particle size in the context of the present invention is defined as the longest dimension of a particle (such i.e. the diameter in case of spherical particle).

All particle sizes are determined by laser diffraction technique using a “Mastersizer 3000” of Malvern Instruments Ltd., UK. Further information on this particle size characterization method can e.g. be found in “Basic principles of particle size analytics”, Dr. Alan Rawle, Malvern Instruments Limited, Enigma Business Part, Grovewood Road, Malvern, Worcestershire, WR14 1XZ, UK and the “Manual of Malvern particle size analyzer”. Particular reference is made to the user manual number MAN 0096, Issue 1.0, November 1994. If nothing else is stated all particle sizes referring are Dv50 values (volume diameter, 50% of the population resides below this point, and 50% resides above this point) determined by laser diffraction. The particle size can be determined in the dry form.

Therefore, the present invention relates to a solid formulation (SF1) comprising
(i) at least one PUFA salt, and
(ii) at least one protein hydrolysate from a starch containing plant, wherein the average particle size is 10-200 μm.

Therefore, the present invention relates to a solid formulation (SF1′) comprising
(i) at least one PUFA salt, and
(ii) at least one protein hydrolysate from a starch containing plant, wherein the average particle size is 200-1000 μm.

Therefore, the present invention relates to a solid formulation (SF1″) comprising
(i) at least one PUFA salt, and
(ii) at least one protein hydrolysate from a starch containing plant, wherein the average particle size above 1000 μm.

The PUFAs are classified according to the position of the double bonds in the carbon chain of the molecule as n-9, n-6 or n-3 PUFAs. Examples of n-6 PUFAs are linoleic acid (C18:2), arachidonic acid (C20:4), γ-linolenic acid (GLA, C18:13) and dihomo-γ-linolenic acid (DGLA, C20:3). Examples of n-3 PUFAs are α-linolenic acid (C18:13), eicosapentaenoic acid (EPA, C20:5), and docosahexaenoic acid (DHA, C22:6). Especially EPA and DHA have attracted interest of the food industry in recent years. The most available sources of these two fatty acids are fish and the marine oils extracted from them. Suitable PUFA salts are the sodium, potassium, magnesium and/or calcium salts. Mixed salts are also suitable.

Very suitable PUFA oils are commercially available for example from DSM Nutritional Products Ltd. These suitable PUFA oils are MEG-3® 4020 EE Oil, MEG-3® 4030 EE Oil, MEG-3® 4421 EE Oil and MEG-3® 5020 EE Oil, which are then transformed into the salts.

Therefore, the present invention relates to a solid formulation (SF2), which is solid formulation (SF), (SF1), (SF1′) or (SF1″), wherein the PUFA salts are the sodium, potassium, magnesium and/or calcium salts.

Therefore, the present invention relates to a solid formulation (SF3), which is solid formulation (SF), (SF1), (SF1′), (SF1″) or (SF2), wherein the PUFA salts are chosen from the group consisting of linoleic acid, arachidonic acid, γ-linolenic acid, dihomoγ-linolenic acid, α-linolenic acid, eicosapentaenoic acid, and docosahexaenoic acid in form of its sodium, potassium, magnesium and/or calcium salts. The most available sources of these two fatty acids are fish and the marine oils extracted from them. Suitable PUFA salts are the sodium, potassium, magnesium and/or calcium salts. Mixed salts are also suitable.

The content of the PUFA salts can vary and it is usually at least 5 weight-% (wt-%), based on the total weight of the solid formulation.

Usually the PUFA salt (or mixture of PUFA salts) are present in an amount of up to 80 wt-%, based on the total weight of the solid formulation.

Preferably, the solid formulation according to the present invention comprises 10-70 wt-%, based on the total weight of the solid formulation, of at least one PUFA salt.

More preferably, the solid formulation according to the present invention comprises 20-60 wt-%, based on the total weight of the solid formulation, of at least one PUFA salt.

Therefore, the present invention relates to a solid formulation (SF4), which is solid formulation (SF), (SF1), (SF1′), (SF1″), (SF2) or (SF3), wherein the solid formulation comprises 5-80 wt-%, based on the total weight of the solid formulation, of at least one PUFA salt.

Therefore, the present invention relates to a solid formulation (SF5), which is solid formulation (SF), (SF1), (SF1′), (SF1″), (SF2), (SF3) or (SF4), wherein the solid formulation comprises 10-70 wt-%, based on the total weight of the solid formulation, of at least one PUFA salt.

More preferably, the solid formulation according to the present invention comprises 20-60 wt-%, based on the total weight of the solid formulation, of at least one PUFA salt.

The solid formulation according to the present also comprises at least one protein hydrolysate, which is from a starch containing plant.

Starch containing plants are i.e. rice, potato, pea, etc.

Especially preferred is rice protein hydrolysate.

The rice protein hydrolysate can be produced using well-known and described methods.

Alternatively, it is also available commercially for example from FrieslandCampina DOMO under the tradename Hyvital® Rice CMA 500.

The content of the protein hydrolysate from a starch containing plant can vary and it is usually at least 10 wt-%, based on the total weight of the solid formulation.

Usually the protein hydrolysate from a starch containing plant is present in an amount of up to 75 wt-%, based on the total weight of the solid formulation.

Furthermore, the present invention relates to a solid formulation comprising 10-75 wt-%, based on the total weight of the solid formulation, of at least one protein hydrolysate from a starch containing plant.

Furthermore, the present invention relates to a solid formulation comprising 15-70 wt-%, based on the total weight of the solid formulation, of at least one protein hydrolysate from a starch containing plant.

In case at least another component is present in the solid formulation the amount of the protein hydrolysate from a starch containing plant is usually lower.

Therefore, the present invention relates to a solid formulation (SF6), which is solid formulation (SF), (SF1), (SF1′), (SF1″), (SF2), (SF3), (SF4) or (SF5), wherein the solid formulation comprising 10-75 wt-%, based on the total weight of the solid formulation, of at least one protein hydrolysate from a starch containing plant.

Therefore, the present invention relates to a solid formulation (SF7), which is solid formulation (SF), (SF1), (SF1′), (SF1″), (SF2), (SF3), (SF4), (SF5) or (SF6), wherein the solid formulation comprising 15-70 wt-%, based on the total weight of the solid formulation, of at least one protein hydrolysate from a starch containing plant.

All percentage in the context of the present invention are always added up to 100% in each solid formulation. In case the PUFA salts(s) and the protein hydrolysate(s) do not add up to 100% there is at least one other ingredient present.

Furthermore, the solid formulation according to the present invention can also comprise further ingredients.

A preferred group of such ingredients are gums, such as xanthan gum, gum arabic, gum ghatti, agar, alginic acid, sodium alginate, carrageenan, gum tragacanth, karaya gum, guar gum, locust bean gum or gellan gum.

These gums can even improve further improve the stability of the solid formulations according to the present invention.

A very preferred gum is gum arabic.

Preferably, the solid formulation comprises up to 30 wt-%, based on the total weight of the solid formulation, of at least one gum.

More preferably, the solid formulation comprises up to 30 wt-%, based on the total weight of the solid formulation, of at least one gum chosen from the group consisting of xanthan gum, gum arabic, gum ghatti, agar, alginic acid, sodium alginate, carrageenan, gum tragacanth, karaya gum, guar gum, locust bean gum or gellan gum. Even more preferably, the solid formulation comprises up to 30 wt-%, based on the total weight of the solid formulation, of gum arabic.

Therefore, the present invention relates to a solid (SF8), which is solid formulation (SF), (SF1), (SF1′), (SF1″), (SF2), (SF3), (SF4), (SF5), (SF6) or (SF7), wherein the solid formulation comprises up to 30 wt-%, based on the total weight of the solid formulation, of at least one gum.

Therefore, the present invention relates to a solid formulation (SF8′), which is solid formulation (SF8), wherein the solid formulation comprises up to 30 wt-%, based on the total weight of the solid formulation, of at least one gum chosen from the group consisting of xanthan gum, gum arabic, gum ghatti, agar, alginic acid, sodium alginate, carrageenan, gum tragacanth, karaya gum, guar gum, locust bean gum or gellan gum.

Therefore, the present invention relates to a solid formulation (SF8″), which is solid formulation (SF8), wherein the solid formulation comprises up to 30 wt-%, based on the total weight of the solid formulation, of gum arabic.

Another preferred group of such ingredients are sugar alcohols (also called polyhydric alcohols, polyalcohols, alditols or glycitols).

Suitable sugar alcohols are for example glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol and polyglycitol.

Very preferred sugar alcohols are mannitol or maltitol.

Preferably, the solid formulation comprises up to 30 wt-%, based on the total weight of the solid formulation, of at least sugar alcohol.

More preferably, the solid formulation comprises up to 30 wt-%, based on the total weight of the solid formulation, of at least one sugar alcohol chosen from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol and polyglycitol.

Even more preferably, the solid formulation comprises up to 30 wt-%, based on the total weight of the solid formulation, of mannitol and/or maltitol.

Therefore, the present invention relates to a solid (SF9), which is solid formulation (SF), (SF1), (SF1′), (SF1″), (SF2), (SF3), (SF4), (SF5), (SF6), (SF7), (SF8), (SF8′) or (SF8″), wherein the solid formulation comprises up to 30 wt-%, based on the total weight of the solid formulation, of at least one sugar alcohol.

Therefore, the present invention relates to a solid formulation (SF9′), which is solid formulation (SF9), wherein the solid formulation comprises up to 30 wt-%, based on the total weight of the solid formulation, of at least one sugar alcohol from the group consisting of glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol and polyglycitol.

Therefore, the present invention relates to a solid formulation (SF9″), which is solid formulation (SF9), wherein the solid formulation comprises up to 30 wt-%, based on the total weight of the solid formulation, of mannitol and/or maltitol.

Furthermore, the solid formulation according to the present invention can further comprise at least one auxiliary agent, wherein the auxiliary agent is chosen from the group consisting of antioxidants (such as ascorbic acid or salts thereof, tocopherol (synthetic or natural); butylated hydroxytoluene (BHT); butylated hydroxyanisole (BHA); propyl gallate; tert. butyl hydroxyquinoline, ascorbic acid esters of a fatty acid and/or ethoxyquin), plasticisers, stabilisers, humectants, protective colloids, dyes, fragrances, fillers and buffers.

These auxiliary agents can be present in an amount of up to 30 wt-%, based on the total weight of the solid formulation.

Therefore the present invention relates to a solid (SF10), which is solid formulation (SF), (SF1), (SF1′), (SF1″), (SF2), (SF3), (SF4), (SF5), (SF6), (SF7), (SF8), (SF8′), (SF8″), (SF9), (SF9′) or (SF9″), wherein the solid formulation comprises at least one auxiliary agent, wherein the auxiliary agent is chosen from the group consisting of antioxidants (such as ascorbic acid or salts thereof, tocopherol (synthetic or natural); butylated hydroxytoluene (BHT); butylated hydroxyani-sole (BHA); propyl gallate; tert. butyl hydroxyquinoline, ascorbic acid esters of a fatty acid and/or ethoxyqu in), plasticisers, stabilisers, humectants, protective colloids, dyes, fragrances, fillers and buffers.

Therefore, the present invention relates to a solid (SF10′), which is solid formulation (SF10), wherein the solid formulation comprises up to 30 wt-%, based on the total weight of the solid formulation of at least one auxiliary agent.

The shape of the particles of the solid formulation according to the present invention is not an essential feature of the present invention. The shape can be sphere-like or any other form (also mixtures of shapes). Usually and preferably, the particles are sphere-like.

One of the main advantages of the solid formulations according to the present invention also lies in the production of the solid formulation.

No emulsification step is needed.

All ingredients are dissolved in water and then usually spray dried. Other drying techniques like spray granulation or beadlet process may be applied as well.

The process of production of the solid formulation is usually the following
(i) the water-soluble ingredients of the matrix are mixed in their dry form and then dissolved in water; afterwards
(ii) the PUFA salt is added, afterwards
(iii) the mixture is (spray) dried.

It is also possible that all water-soluble ingredients are solved in water (and not mixed in dry form).

Depending on the temperature of the spray drying process, the solid formulation can still comprise water (usually not more than 5 wt-%, based on the total weight of the solid formulation).

The solid formulation (SF), (SF1), (SF1′), (SF1″), (SF2), (SF3), (SF4), (SF5), (SF6), (SF7), (SF8), (SF8′), (SF8″), (SF9), (SF9′), (SF9″), (SF10) or (SF10′) can also be used as such or used to be incorporated into other product forms.

The solid formulation can be incorporated into food, feed, pharmaceutical and/or personal care products.

The solid formulation can be also being incorporated into a premix. This premix is then incorporated into a food, feed, pharmaceutical and/or personal care product.

Another embodiment of the present invention is the use of at least one protein hydrolysate from a starch containing plant for manufacturing a solid formulation comprising at least one PUFA salt.

The solid formulation according to the present invention can also be used in pharmaceutical products. The pharmaceutical product can be in any galenical form, usually in the form of tablets.

A further embodiment of the present invention relates to food products, feed products, dietary supplements and/or pharmaceutical products, comprising at least one solid formulation (SF), (SF1), (SF1′), (SF1″), (SF2), (SF3), (SF4), (SF5), (SF6), (SF7), (SF8), (SF8′), (SF8″), (SF9), (SF9′), (SF9″), (SF10) and/or (SF10′).

The invention is illustrated by the following Examples. All temperatures are given in ° C. and all parts and percentages are related to the weight.

General

All the solid formulations, which were tested, are produced using the procedure described in Example 1. The ingredients and/or their concentration vary, not the process parameters.

The salts (Na or K) of the following commercially available PUFA oils (available from DSM Nutritional Products Ltd) have been used
MEG-3® 4020 EE Oil (This s a marine omega-3 long chain polyunsaturated fatty acid. This ethyl ester has a minimum of 360 mg EPA, 180 mg DHA, and 590 mg total Omega-3 per gram).
MEG-3® 4030 EE Oil (this is a marine omega-3 long chain polyunsaturated fatty acid. This ethyl ester has a minimum of 360 mg EPA, 270 mg DHA, and 680 mg total Omega-3 per gram.)
MEG-3® 4421 EE Oil is a marine omega-3 long chain polyunsaturated fatty acid. This ethyl ester has a minimum of 400 mg EPA, 200 mg DHA, and 650 mg total Omega-3 per gram.
MEG-3® 5020 EE Oil is a marine omega-3 long chain polyunsaturated fatty acid. This ethyl ester has a minimum of 460 mg EPA, 180 mg DHA, and 700 mg total Omega-3 per gram

### EXAMPLE 1

25 g of maltodextrin (maltodextrin 28-31), 20 g of sodium ascorbate and 80 g of rice hydrolysate (Hyvital® Rice CMA 500 from FrieslandCampina Domo) were put (in their dry state) into a beaker and mixed well.

Afterward 800 g of water were added slowly to this mixture under constant stirring. This solution was heated up to 50° C., and adjust the pH was adjusted (by NaOH or KOH) to 8.5.

The PUFA salt (the Na salt of MEG-3® 4030 EE Oil) was also heated up to 50° C. and then the warm PUFA salt was added to the aqueous solution. A slurry way obtained.

The so obtained slurry was spray dried (using a GEA MOBILE MINOR™), inlet temperature was set at 150˜180° C., outlet temperature was controlled around 60˜80° C. A free-flowing powder was obtained.

Testing of the Solid Formulations

The storage stability of the produced solid formulations was tested as follows:

The solid formulations were stored at room temperature and after defined storage times the formulations were evaluated by a sensory panel of experienced and well-trained persons.

Each person of this panel sniffed at the solid formulations and gave them a value of the sensory scale.

This sensory scale, which was applied, has values that goes from 0 to 15.0 means no smell 15 means extremely strong.

The following compositions are tested (the amount of the ingredients is given in gram (g)):

TABLE 1
Formulations (1-2). The formulation 1 is the
one produced as in Example 1. The form 2 is
produced according to the method of example 1.

| Ingredients | FORM 1 | FORM2 |
|---|---|---|
| PUFA Na Salt of MEG- | 250 | 250 |
| 3 ® 4030 EE Oil | | |
| Maltodextrin 28-31 | 25 | 25 |
| Sodium Ascorbate | 15 | 20 |
| Rice hydrolysate | 80 | 130 |
| Water | 1000 | 1000 |

TABLE 2
the sensory results of the forms 1-2

| | | | Fishy Marine Complex | Other Off |
|---|---|---|---|---|
| FORM 1 | initial | | 0.8 | |
| | 4 | weeks | 1.0 | 0.5 |
| | 8 | weeks | | 1.0 |
| | 12 | weeks | | 1.0 |
| | 16 | weeks | 0.8 | |
| | 20 | weeks | 0.8 | |
| FORM 2 | initial | | | |
| | 4 | weeks | | 4.0 |
| | 8 | weeks | | 2.5 |
| | 12 | weeks | | 3.0 |
| | 16 | weeks | | 3.5 |
| | 20 | weeks | | 1.5 |

Formulations 3 and 4:

The formulations are produced in accordance with the process as disclosed in Example 1.

TABLE 3
formulations 3 and 4

| Ingredients | FORM 3 | FORM 4 |
|---|---|---|
| PUFA Na Salt 40:30 | 250 | 250 |
| Maltodextrin 28-31 | 25 | 65 |
| Sodium Ascorbate | 20 | 20 |
| Rice hydrolysate | 60 | 60 |
| TIC Pretested ® Gum Arabic | 70 | |
| Spray Dry Powder - Grade #1 | | |
| Alginate LFR 5/60 | | 30 |
| Water | 1000 | 1000 |

Sensory Results

TABLE 4
sensory results of formulations 3 and 4

| | | | Fishy Marine Complex | Other Off |
|---|---|---|---|---|
| FORM 3 | initial | | | |
| | 4 | weeks | | |
| | 8 | weeks | | |
| | 12 | weeks | | 0.8 |
| | 16 | weeks | | |
| | 20 | weeks | | |
| FORM 4 | initial | | 1.0 | |
| | 4 | weeks | 2.0 | |
| | 8 | weeks | | |
| | 12 | weeks | | 0.8 |
| | 16 | weeks | | |
| | 20 | weeks | | |

Formulations 5 and 6:

TABLE 5
formulations 5 and 6 (these are comparative examples)

| Ingredient | FORM 5-9 | FORM 6 |
|---|---|---|
| PUFA Na Salt 40:30 | 250 | 250 |
| Maltodextrin 28-31 | 25 | 25 |
| Sodium Ascorbate | 15 | 15 |
| Supro 313 | 80 | |
| Soy isolate Partially hydro | | |
| Supro 670 | | 80 |
| Soy isolate Partially hydro | | |

Sensory Results

TABLE 6
sensory results of formulations 5 and 6

| | | Fishy Marine Complex | Other Off |
|---|---|---|---|
| FORM 5 | initial | 3 | |
| | 4 weeks | 5 | |
| | 8 weeks | 4 | |
| FORM 6 | initial | 4 | |
| | 4 weeks | 4 | |
| | 8 weeks | 5 | |

Formulation 7:

TABLE 7
formulation 7 (comparative example)

| Ingredient | FORM 7 |
|---|---|
| PUFA Na Salt 40:30 | 250 |
| Maltodextrin 28-31 | 25 |
| Sodium Ascorbate | 15 |
| Cottonseed hydrolysate | 80 |
| Water | 1000 |

Sensory Results

TABLE 6
sensory results of formulation 7

| | | Fishy Marine Complex | Other Off |
|---|---|---|---|
| FORM 7 | initial | 0 | |
| | 4 weeks | 4 | |

It can be seen from these evaluation tests that the solid formulations according to the present invention are better significantly than such, which are produced with a different (commonly and widely used) matrix material.

1. A solid formulation comprising
(i) at least one PUFA salt, and
(ii) at least one protein hydrolysate from a starch containing plant.

2. Solid formulation according to claim 1,
wherein the average particle size (Dv50) of the solid formulation is 10-200 μm.

3. Solid formulation according to claim 1,
wherein the average particle size (Dv50) of the solid formulation is 200-1000 μm.

4. Solid formulation according to claim 1,
wherein the average particle size (Dv50) of the solid formulation is more than 1000 μm.

5. Solid formulation according to claim 1, wherein the PUFA salts are the sodium, potassium, magnesium and/or calcium salts.

6. Solid formulation according to claim 1, wherein the PUFA salts are chosen from the group consisting of linoleic acid, arachidonic acid, γ-linolenic acid, dihomo-γ-linolenic acid, α-linolenic acid, eicosapentaenoic acid, and docosahexaenoic acid in form of its sodium, potassium and/or calcium salts.

7. Solid formulation according to claim 1, wherein the solid formulation comprises 5-80 wt-%, based on the total weight of the solid formulation, of at least one PUFA salt.

8. Solid formulation according to claim 1, wherein the solid formulation comprising 10-75 wt-%, based on the total weight of the solid formulation, of at least one protein hydrolysate from a starch containing plant.

9. Solid formulation according to claim 1, wherein the solid formulation comprises up to 30 wt-%, based on the total weight of the solid formulation, of at least one gum.

10. Solid formulation according to claim 1, wherein the solid formulation comprises up to 30 wt-%, based on the total weight of the solid formulation, of at least one sugar alcohol.

11. Solid formulation according to claim 1, wherein the solid formulation comprises at least one auxiliary agent, wherein the auxiliary agent is chosen from the group consisting of antioxidants chosen from the groups consisting of ascorbic acid or salts thereof, synthetic tocopherol, natural tocopherol, butylated hydroxytoluene, butylated hydroxyanisole, propyl gallate; tert. butyl hydroxyquinoline, ascorbic acid esters of a fatty acid and ethoxyquin; plasticisers, stabilisers; humectants chosen from the group consisting of glycerine, sorbitol and polyethylene glycol; protective colloids; dyes; fragrances; fillers and buffers.

12. Process of production of the solid formulation wherein
(i) the water soluble ingredients of the matrix are mixed in their dry form and then dissolved in water; afterwards
(ii) the PUFA salt is added, afterwards
(iii) the mixture is spray dried.

13. Food products, feed products, dietary supplements, pharmaceutical products and/or premixes, comprising at least one solid formulation according to claim 1.

14. Use of at least one protein hydrolysate from a starch containing plant for manufacturing a solid formulation comprising at least one PUFA salt.
